(19) United States
(12) Patent Application Publication
(10) Pub. No.: US 2023/0372670 A1
(43) Pub. Date: Nov. 23, 2023

(54) WIRE SUPPORTED EXPANDABLE CATHETER TIP

(71) Applicant: Neuravi Limited, Galway (IE)

(72) Inventor: Karl KEATING, Galway (IE)

(73) Assignee: Neuravi Limited, Galway (IE)

(21) Appl. No.: 18/127,323

(22) Filed: Mar. 28, 2023

Related U.S. Application Data
(60) Provisional application No. 63/344,073, filed on May 20, 2022.

(51) Int. Cl. A61M 25/01; A61M 25/00

(52) U.S. Cl. CPC A61M 25/01; A61M 25/0067

(57) ABSTRACT

The designs disclosed herein are for a clot retrieval catheter with a large bore shaft and an expandable distal tip section. The catheter includes proximal elongate shaft comprising a distal end, a longitudinal axis, and a shaft braid member comprising a plurality of braid wires. The expandable distal tip section is located proximate the distal end of the proximal elongate shaft. The expandable distal tip sections can vary in design. In one example, the distal tip section includes a longitudinal array of hoops. The plurality of braid wires can be formed monolithically with the array of hoops. In another example, the distal tip section can include two sets of opposing ribs. A first rib can be formed from a first wire of the plurality of braid wires, and a second rib can be formed from a second wire of the plurality of braid wire.

### CROSS-REFERENCE TO RELATED APPLICATION

The present application claims benefit of priority to U.S. Provisional Patent Application No. 63/344,073 filed May 20, 2022. The entire contents of which are hereby incorporated by reference.

### FIELD OF INVENTION

The present invention generally relates to devices and methods for removing acute blockages from blood vessels during intravascular medical treatments. More specifically, the present invention relates to retrieval catheters with expandable tips into which an object or objects can be retrieved.

### BACKGROUND

There are significant challenges associated with designing clot removal devices that can deliver high levels of performance. For example, in cases where access involves navigating the aortic arch (such as coronary or cerebral blockages), the configuration of the arch in some patients makes it difficult to position a guide catheter. These difficult arch configurations are classified as either type 2 or type 3 aortic arches, with type 3 arches presenting the most difficulty. The tortuosity challenge is even more severe in the arteries approaching the brain. For example it is not unusual at the distal end of the internal carotid artery that the device will have to navigate a vessel segment with a 180° bend, a 90° bend and a 360° bend in quick succession over a few centimeters of vessel. In the case of pulmonary embolisms, access may be gained through the venous system and then through the right atrium and ventricle of the heart. The right ventricular outflow tract and pulmonary arteries are delicate vessels that can easily be damaged by inflexible or high profile devices.

The vasculature in the area in which the clot may be lodged is often fragile and delicate. For example neurovascular vessels are more fragile than similarly sized vessels in other parts of the body and are in a soft tissue bed. Excessive tensile forces applied on these vessels could result in perforations and hemorrhage. Pulmonary vessels are larger than those of the cerebral vasculature, but are also delicate in nature, particularly those more distal vessels. The clots may not only range in shape and consistency, but also may vary greatly in length, even in any one given area of the anatomy. For example, clots occluding the middle cerebral artery of an ischemic stroke patient may range from just a few millimeters to several centimeters in length.

Stent-like clot retrievers are being increasingly used to remove clot from cerebral vessels of acute stroke patients. These are self-expanding devices, similar in appearance to a stent attached to the end of a long shaft, and are advanced through a microcatheter and deployed across clot obstructions in order to trap and retrieve them. They rely on a pinning mechanism to grab the clot by trapping the clot between the self-expanding stent-like body and the vessel wall. This approach has a number of disadvantages. A stent-like clot retriever relies on its outward radial force (RF) to retain its grip on the clot. If the RF is too low the stent-like clot retriever will lose its grip on the clot, but if the RF is too high the stent-like clot retriever may damage the vessel wall and may require too much force to withdraw. Therefore stent-like clot retrievers that have sufficient radial force to deal with all clot types may cause vessel trauma and serious patient injury, and stent-like clot retrievers that have appropriate radial force to remain atraumatic may not be able to effectively handle all clot types.

Conventional stent-like clot retriever designs do not retain their expanded shape very well when placed in tension in bends, due to the manner in which their strut elements are connected to one another. This can result in a loss of grip on a clot as the stent-like clot retriever is withdrawn proximally around a bend in a tortuous vessel, with the potential escape of the captured clot. This occurs because the struts of the stent-like clot retriever are placed in tension when it is retracted. This tension is due to friction between the device and the blood vessel, and is increased if an additional load is applied load such as that provided by a clot. In a bend the struts on the outside of the bend are placed in higher tension than those on the inside. In order to attain the lowest possible energy state the outside surface of the stent moves towards the inside surface of the bend, which reduces the tension in the struts, but also reduces the expanded diameter of the stent-like clot retriever.

The challenges described above need to be overcome for any device to provide a high level of success in removing clot, restoring flow and facilitating good patient outcomes. Existing devices do not adequately address these challenges.

### SUMMARY

It is an object of the present designs to provide devices and methods to meet the above-stated needs. The designs described herein can include a wire supported expandable catheter. The catheter can include a proximal elongate shaft including a distal end, a longitudinal axis, and a shaft braid member having a plurality of braid wires. The catheter can include a distal tip section at the distal end of the elongate shaft. The distal tip section can have a collapsed delivery configuration, an expanded deployed configuration, and a longitudinal array of hoops. The plurality of braid wires of the proximal elongate shaft can be formed monolithically with the array of hoops of the distal tip section. Each wire of the plurality of braid wires can diverge from a final crossover point of the shaft braid member at the distal end of the proximal elongate shaft to form one of the hoops of the longitudinal array of hoops.

In some examples, the distal tip section can include a collapsed inner diameter in the collapsed delivery configuration, which can be less than an expanded inner diameter in the expanded deployed configuration.

In some examples, the wire of each hoop of the longitudinal array of hoops can be connected to the elongate shaft at the final crossover point by a pair of axially extending hoop runners. In some examples, each pair of axially extending hoop runners can be spaced evenly around the longitudinal axis. In some examples, each hoop of the longitudinal array of hoops can diverge radially from a pair of hoop termini at the distal end of each pair of axially externing hoop runners to extend circumferentially around the tip section.

In some examples, a first spacing between a pair of more proximal adjacent hoops of the longitudinal array of hoops can be different than a second spacing between adjacent hoops in a more distal pair of adjacent hoops. In some examples, each hoop of the longitudinal array of hoops can include a distally unconnected peak which moves distally when the distal tip section is folded to the collapsed delivery configuration.

In some examples, each hoop of the longitudinal array of hoops can define a plane that is normal to the longitudinal axis of the elongate shaft.

In some examples, the longitudinal array of hoops defining a mouth with a curvilinear profile.

The designs described herein include a catheter. The catheter can include a proximal elongate shaft having a distal end, a longitudinal axis, and a shaft braid member having a plurality of braid wires. The catheter can include a distal tip section extending from the distal end of the elongate shaft. The distal tip section can include two sets of opposing ribs. A first rib from the two sets of opposing ribs can be formed from a first wire of the plurality of braid wires of the proximal elongate shaft, and a second rib from the two sets of opposing ribs can be formed from a second wire of the plurality of braid wires of the proximal elongate shaft. The first rib can be spaced approximately 180 degrees about the longitudinal axis from the second rib.

In some examples, the distal tip section can include a delivery configuration and a clot capture configuration. The distal tip section can have a smaller delivery inner diameter in the delivery configuration and a larger expanded inner diameter when impinged radially by an ingested clot in the clot capture configuration.

In some examples, the distal tip section can include a collapsed delivery configuration having a collapsed inner diameter and an expanded deployed configuration heat set to have an expanded inner diameter greater than the collapsed inner diameter.

In some examples, each rib of the two sets of opposing ribs can have a distally unconnected peak.

In some examples, the two sets of opposing ribs can be formed monolithically with the plurality of braid wires of the shaft braid member.

In some examples, each wire of the plurality of braid wires can diverge radially from a final crossover point to form a v-shaped pattern.

In some examples, the sets of opposing ribs can define a mouth with a curvilinear profile.

The designs described herein can include a catheter. The catheter can include a proximal elongate shaft having a distal end, a longitudinal axis, and a shaft braid member including a plurality of braid wires. The catheter can include a distal tip section at the distal end of the elongate shaft. The distal tip section can have a longitudinal array of offset hoops defining a beveled plane. The catheter can include a distal outer jacket surrounding the longitudinal array of offset hoops. The plurality of braid wires of the proximal elongate shaft can be formed monolithically with the array of offset hoops. Each wire of the plurality of braid wires can diverge from a final crossover point to extend circumferentially around the tip section. The beveled plane can cross the longitudinal axis at an acute angle.

In some examples, the distal tip section can have a collapsed delivery configuration having a collapsed inner diameter and an expanded deployed configuration having an expanded inner diameter heat set to be greater than the collapsed inner diameter.

In some examples, the distal tip section can have a larger expanded inner diameter when impinged radially by an ingested clot in the expanded clot capture configuration and a smaller delivery inner diameter in the delivery configuration. In some examples, the distal tip section can have a substantially circular cross section with a center radially offset from the longitudinal axis of the elongate shaft, when in the expanded deployed configuration.

In some examples, the distal tip section can have a substantially circular cross section with a center substantially coincident with the longitudinal axis of the elongate shaft, when in the expanded deployed configuration.

In some examples, the array of offset hoops can have a distally unconnected peaks which move distally when the distal tip section is folded to the collapsed delivery configuration.

In some examples, the array of offset hoops following a curvilinear profile circumferentially around the tip section.

In some examples, at least a portion of a perimeter of one hoop of the array of offset hoops can define a plane passing through the longitudinal axis at an acute angle.

In some examples, the distal tip section can include a distal expansile section configured to expand radially when ingesting a clot.

In some examples, the array of offset hoops can define a mouth with a curvilinear profile.

Other aspects of the present disclosure will become apparent upon reviewing the following detailed description in conjunction with the accompanying figures. Additional features or manufacturing and use steps can be included as would be appreciated and understood by a person of ordinary skill in the art.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation. It is expected that those of skill in the art can conceive of and combine elements from multiple figures to better suit the needs of the user.

FIG. 1 is a view of a clot retrieval catheter with an expandable tip being advanced through the vasculature, according to aspects of the present invention;

FIG. 2 illustrates the distal portion of a clot retrieval catheter with an expandable tip, according to aspects of the present invention;

FIG. 3 illustrates the distal portion of a clot retrieval catheter with an expandable tip, according to aspects of the present invention;

FIG. 4 is an end, elevation view of the distal portion of the clot retrieval catheter of FIG. 3, according to aspects of the present invention;

FIG. 5 illustrates the distal portion of a clot retrieval catheter with an expandable tip that opens into a beveled plane, according to aspects of the present invention;

FIG. 6 is an end, elevation view of the distal portion of the clot retrieval catheter of FIG. 5, according to aspects of the present invention;

FIG. 7 is a schematic of a catheter having a symmetrically-opening distal portion, according to aspects of the present invention;

FIG. 8 is a schematic of a catheter having an asymmetrically-opening distal portion, according to aspects of the present invention;

FIGS. 9A-9C are schematics illustrating a self-expanding catheter used to capture a clot, according to aspects of the present invention;

FIG. 10 illustrates the distal portion of a clot retrieval catheter with an expandable tip, according to aspects of the present invention;

FIG. 11 illustrates the distal portion of a clot retrieval catheter with an expandable tip, according to aspects of the present invention;

FIG. 12 is a cutaway view of the clot retrieval catheter of FIG. 11, which shows the detail of an outer jacket, according to aspects of the present invention; and

FIGS. 13A-13C are schematics illustrating a non-self-expanding catheter used to capture a clot, according to aspects of the present invention.

### DETAILED DESCRIPTION

Specific examples of the present invention are now described in detail with reference to the Figures, where identical reference numbers indicate elements which are functionally similar or identical. The examples address many of the deficiencies associated with traditional clot retrieval aspiration catheters, such as poor or inaccurate deployment to a target site and ineffective clot removal.

The designs herein can be for a clot retrieval catheter with a large internal lumen and a distal funnel tip that can self-expand to a diameter larger than that of the guide or sheath through which it is coaxially delivered. The designs can have a proximal elongate shaft for the shaft of the catheter, and a distal tip with an expanding braided support structure and outer polymeric jacket to give the tip atraumatic properties. The braided support can be designed so that the expansion capability is variably focused in an axial portion of the tip section. The braid can be capable of easily and repeatably collapsing for delivery and expanding for good clot reception and resistance under aspiration. The catheter's braid and tip designs can be sufficiently flexible to navigate highly tortuous areas of the anatomy and be able to recover its shape to maintain the inner diameter of the lumen when displaced in a vessel.

Accessing the various vessels within the vascular, whether they are coronary, pulmonary, or cerebral, involves well-known procedural steps and the use of a number of conventional, commercially-available accessory products. These products, such as angiographic materials, mechanical thrombectomy devices, microcatheters, and guidewires are widely used in laboratory and medical procedures. When these products are employed in conjunction with the devices and methods of this invention in the description below, their function and exact constitution are not described in detail. Additionally, while the description is in many cases in the context of thrombectomy treatments in intercranial arteries, the disclosure may be adapted for other procedures and in other body passageways as well.

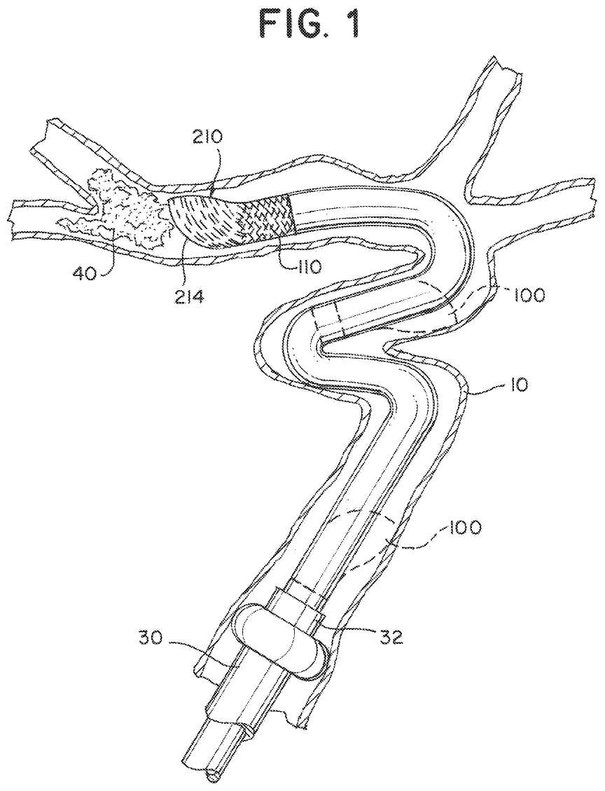

Turning to the figures, FIG. 1 illustrates a possible sequence for approaching an occlusive clot 40 using a clot retrieval catheter 100 of the designs disclosed herein. The clot 40 can be approached with the catheter 100 collapsed within a guide sheath 30 or other outer catheter for delivery. When the vasculature 10 becomes too narrow and/or tortuous for further distal navigation with the guide sheath 30, the catheter 100 can be deployed for further independent travel distally. The catheter 100 can be highly flexible such that it is capable of navigating the M1 or other tortuous regions of the neurovascular to reach an occlusive clot.

The clot retrieval catheter 100 can have a flexible elongate shaft 110 serving as a shaft with a large internal bore (which in some cases can be 0.070 inches or larger) and a distal tip section 210 (see also tips 310, 410, and 510 described herein) having a collapsible supporting braided structure. The large bore helps the catheter to be delivered to a target site by a variety of methods. These can include over a guidewire, over a microcatheter, with a dilator/access tool, or by itself.

In most cases, the design of the collapsible funnel tip can be configured so that the catheter 100 can be delivered through (and retrieved back through) commonly sized outer sheaths and guides. For example, a standard 6Fr sheath/8Fr guide, would typically have an inner lumen of less than 0.090 inches. The tip can then be designed with a collapsed delivery outer diameter of approximately 0.086 inches. The tip can self-expand once advanced to an unconstrained position distal to the distal end 32 of the guide sheath 30, capable of reaching expanded outer diameters as large as approximately 0.132 inches. As the catheter can be delivered independently to a remote occlusion, the tip section 210 (see also tips 310, 410, and 510 described herein) must be designed to be able to resist collapse from the forces of aspiration, have excellent lateral flexibility in both the expanded and collapsed states, and have an atraumatic profile to prevent snagging on bifurcations in vessels.

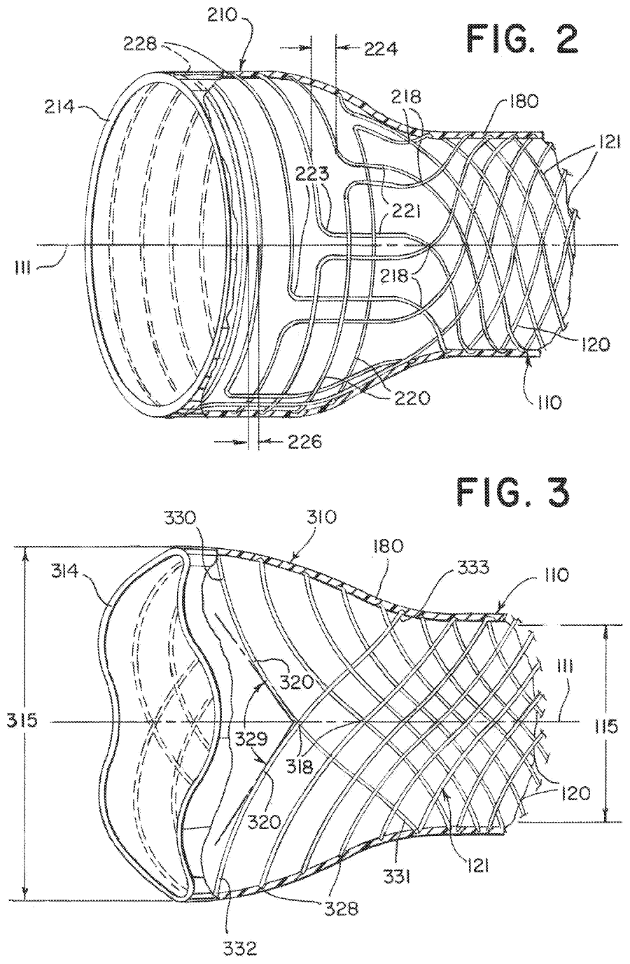

A closer view of the distal portion of the catheter 100 with the tip section 210 in the expanded deployed configuration as a funnel is illustrated in FIG. 2. The elongate shaft 110 can extend along longitudinal axis 111. The distal end of the elongate shaft 110 can have a distal end 114, and the distal end 114 can be proximate the tip section 210. The elongate shaft 110 can include a braid member 120. The braid member 120 can be formed from a plurality of braid wires 121 wrapping the length of the elongate shaft 110. The plurality of braid wires 121 of the braid member 120 can serve as the backbone and support for the catheter 100 shaft. The interlacing weave of the plurality of braid wires 121 can be any number of materials or patterns known in the art and can have a varied density and composition along the length of the shaft. The distal tip section 210 at the distal end 114 of the elongate shaft 110 can have a collapsed delivery configuration and an expanded deployed configuration.

The distal tip section 210 can further include a longitudinal array of hoops 220 extending around the distal tip section 210. This longitudinal array of hoops 220 (also referred to herein as hoops 220 or an array of hoops 220) can be formed continuously from the plurality of braid wires 121. For example, the plurality of braid wires 121 can be formed monolithically with the array of hoops 220 of the distal tip section 210 such that the individual braid wires 121 extend continuously from the elongate shaft 110 to the distal tip section 210. Each wire of the plurality of braid wires 121 can diverge from a final crossover point 218 of the shaft braid member 120 to form one of the hoops 220. In some examples, the catheter 100 can include hoop runners 221 extending between the final crossover point 218 and the hoops 220. For example, at the final crossover point 218 for two braid wires 121, the two braid wires 121 can turn longitudinally along the longitudinal axis 111 of the and extend a certain distance as two hoop runners 221 before diverging to form a single hoop 220 that extends circumferentially around the tip section 210. This points of divergence of the two braid wires 121 to form the hoop 220 can be called hoop termini 223. Referring again to the hoop runners 221, the hoop runners 221 can provide a degree of pushability for the distal tip 210, since their longitudinal direction can resist axial force as the catheter 110 is pushed through a target vessel. In some examples, each pair of axially extending hoop runners 221 can be spaced evenly, and circumferentially, around the longitudinal axis 111.

Referring to the array of hoops 220 of the distal tip section 210, the spacing between two adjacent hoops can vary depending on where the individual hoops are positioned on the distal tip section 210. To illustrate, a first spacing 224 between a pair of more proximal adjacent hoops of the longitudinal array of hoops 220 can be different than a second spacing 226 between adjacent hoops in a more distal pair of adjacent hoops 220. In some examples, the second spacing 226 of adjacent hoops 220 that are near the distal end 214 of the distal tip section 210 can be tighter than the first spacing 224 of adjacent hoops 220 that are more proximal. The tighter spacing at the distal end 214 of the distal tip section 210 can help improve clot retention and reduce crushability of the distal tip section 210 when it is in the expanded deployed configuration. In the expanded deployed configuration, each hoop of the longitudinal array of hoops 220 can define a plane that is normal to the longitudinal axis 111 of the elongate shaft 110. Further, the longitudinal array of hoops 220 can form a series of rings concentric with the longitudinal axis 111 when the distal tip section 210 is in the expanded deployed configuration.

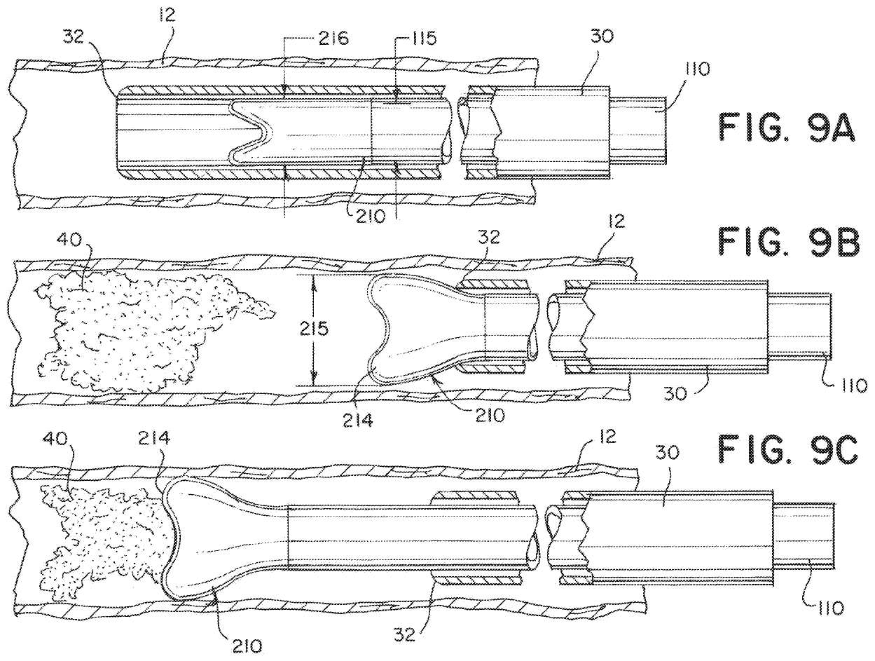

As described above, the distal tip section 210 can have an expanded deployed configuration and a collapsed delivery configuration. As will be described in greater detail with reference to FIGS. 9A-9C, the distal tip section 210 can have a collapsed inner diameter 216 in the collapsed delivery configuration that is less than an expanded inner diameter 215 in the expanded deployed configuration. Each hoop of the longitudinal array of hoops 220 can include a distally unconnected peak 228 which moves distally when the distal tip section 210 is folded to the collapsed delivery configuration.

FIG. 3 illustrates the distal portion of a clot retrieval catheter 100 with an expandable tip 310, according to aspects of the present invention. The design shown in FIG. 3 is similar to that shown in FIG. 2, but with variations to the shape and configuration of the distal tip section 310 (referred to as distal tip section 210 in FIG. 2). The example shown in FIG. 3 can similarly include a proximal elongate shaft 110 having a distal end 114, a longitudinal axis 111, and a shaft braid member 120 comprising a plurality of braid wires 121. The distal tip section 310 can extend from the distal end 114 of the elongate shaft 110. In the example shown in FIG. 3, the distal tip section 310 can include two sets of opposing ribs 320 that flare outward (e.g., open like a book) from each other when the distal tip section 310 is in the expanded configuration. A first rib 330 from the two sets of opposing ribs 320 can be formed from a first wire 331 of the plurality of braid wires 121 of the proximal elongate shaft 110. A second rib 332 from the two sets of opposing ribs 320 can be formed from a second wire 333 of the plurality of braid wires 121 of the proximal elongate shaft 110. The first rib 330 can be spaced approximately 180 degrees about the longitudinal axis 111 from the second rib 332.

The plurality of braid wires 121 can be formed monolithically with the sets of opposing ribs 320. For example, the individual braid wires 121 can extend continuously from the elongate shaft 110 to the distal tip section 310. The braid wires 121 of the proximal elongate shaft 110 can diverge when transitioning into the distal tip section 310. Each wire of the plurality of braid wires 121 can diverge radially from a final crossover point 318 to form a v-shaped pattern 329. After the final crossover point 318, the first rib 330 and the second rib 332 can extend partially around the distal tip section 310. Each rib of the two sets of opposing ribs 320 can include a distally unconnected peak 228 at the distal end 314 of the distal tip section 310, as similarly shown in FIG. 2.

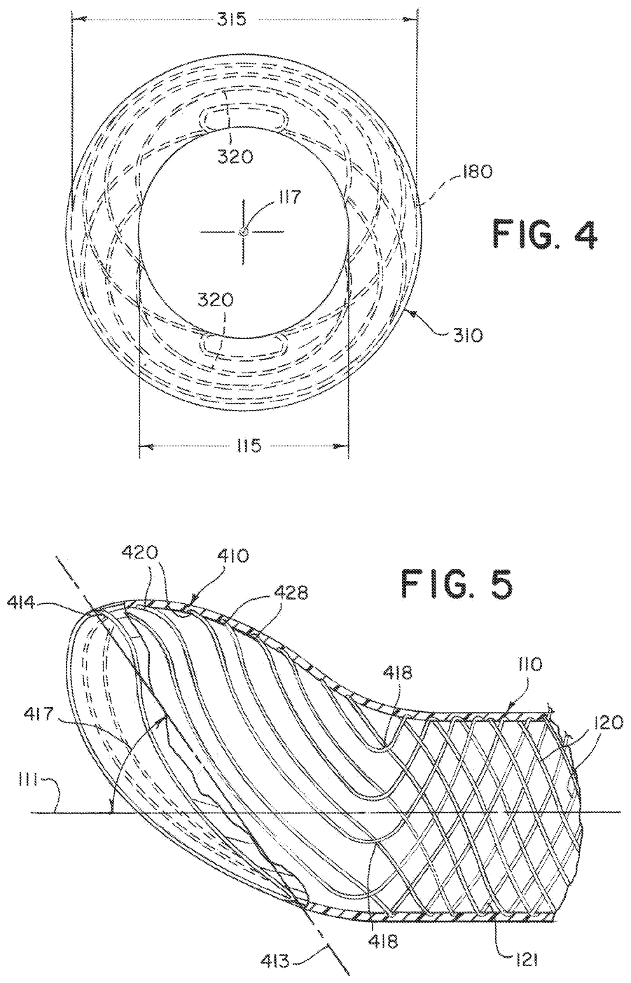

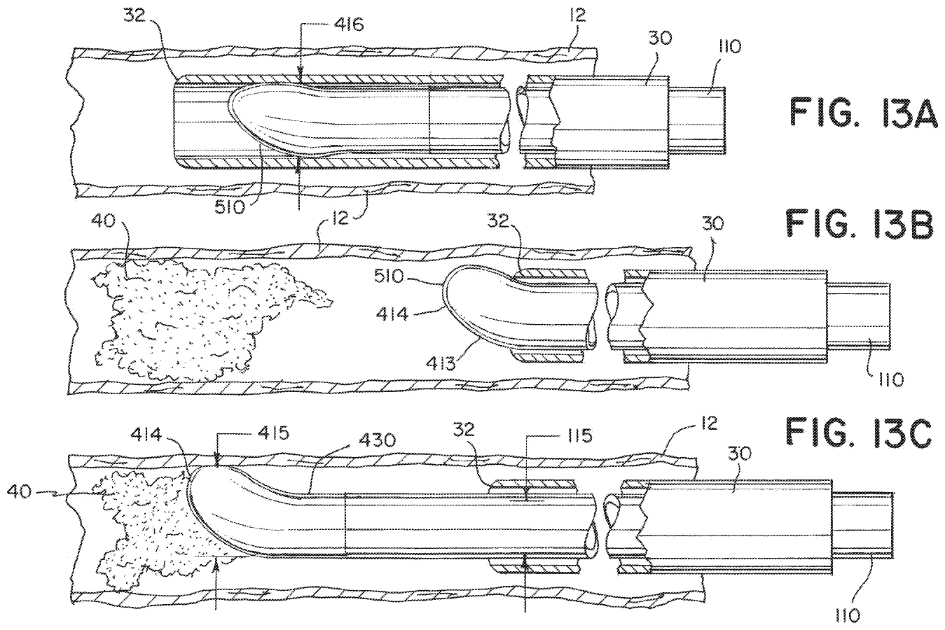

FIG. 4 is an end, elevation view of the distal portion of the clot retrieval catheter of FIG. 3, according to aspects of the present invention. The figure provides a view looking into the expanded distal tip section 310 and into the elongate shaft 110. The distal tip section 310 can have a delivery configuration and a clot capture configuration. The distal tip section 310 can have a smaller delivery inner diameter 115 in the delivery configuration and a larger expanded inner diameter 315 when impinged radially by an ingested clot 40 in the clot capture configuration. In this example, the distal tip section 310 can expand upon making contact with a clot. An example of this configuration is shown in FIGS. 13A-13C. Alternatively, the distal tip section 310 can have a collapsed delivery configuration having a collapsed inner diameter 216 and an expanded deployed configuration. The deployed configuration can be achieved by heat setting the material of the opposing ribs 320 to have an expanded inner diameter 215 greater than the collapsed inner diameter 216. An example of this configuration is shown in FIGS. 9A-9C.

FIG. 5 illustrates the distal portion of a clot retrieval catheter 100 with an expandable tip that opens into a beveled plane 413, according to aspects of the present invention. The catheter 100 can have a proximal elongate shaft 110 comprising a distal end 114, a longitudinal axis 111, and a shaft braid member 120 comprising a plurality of braid wires 121, similar to the designs shown in FIGS. 2-4. The catheter 100 can include a distal tip section 410 at the distal end 114 of the elongate shaft 110. The distal tip section 410 is similar to the distal tip sections described above (e.g., distal tip section 210 and 310), but with variations to the shape and design. The distal tip section 410 can include a longitudinal array of offset hoops 420 defining a beveled plane 413. The beveled plane 413 crossing the longitudinal axis 111 can form at an acute angle 417 such that a distalmost tip 414 of the distal tip section 410 is directed toward the target (e.g., clot).

The distal tip 410 is offset from the longitudinal axis 111, which can help to reduce the likelihood of tip collapse during aspiration compared to expandable catheters with mouths normal to the longitudinal axis 111, since the membrane (e.g., outer jacket 180) for the offset mouth does not fully extend around the diameter of the distal tip section 410. The catheter 100 can further include the outer jacket 180 surrounding the longitudinal array of offset hoops 420. The plurality of braid wires 121 of the proximal elongate shaft 110 can be formed monolithically with the array of offset hoops 420. Each wire of the plurality of braid wires 121 can diverge from a final crossover point 418, wherein beyond the final crossover point 418 the plurality of braid wires 121 extend circumferentially around the tip section 410. Stated otherwise, the plurality of braid wires 121 can discontinue their braided orientation at the final crossover point 418, bend, and continue distally to follow a curvilinear profile circumferentially around the distal tip section 410. The array of offset hoops 420 can include distally unconnected peaks 428 which move distally when the distal tip section 210 is folded to the collapsed delivery configuration.

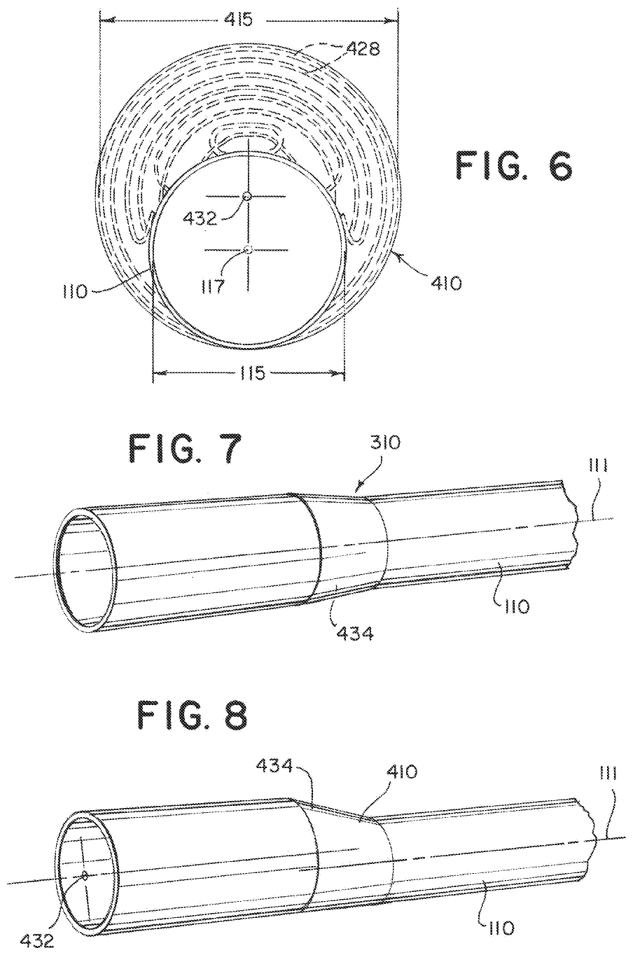

FIG. 6 is an end, elevation view of the distal portion of the clot retrieval catheter 100 of FIG. 5, according to aspects of the present invention. The end view shows the offset of the distal tip section 410. In the expanded deployed configuration, the distal tip section 410 includes a substantially circular cross section with a center 432 radially offset from the longitudinal axis 111 of the elongate shaft 110, i.e., the center 432 of the distal tip section 410 is offset from the center 117 of the elongate shaft 110. The distal tip section 410 can have a collapsed delivery configuration having a collapsed inner diameter 115 approximately the diameter of the elongate shaft 110. The distal tip section 410 can have an expanded deployed configuration having an expanded inner diameter 415 heat set to be greater than the collapsed inner diameter 115.

FIG. 7 is a schematic of a catheter 100 having a symmetrically-opening distal portion, according to aspects of the present invention. The figure shows that, in some examples described herein, the distal tip section 310 can have a substantially circular cross section with a center 432 substantially coincident with the longitudinal axis 111 of the elongate shaft 110, when the catheter 100 is in the collapsed delivery configuration or the expanded deployed configuration. This is shown in the examples in FIGS. 3 and 4 (i.e., distal tip section 310) and in FIG. 2 (i.e., distal tip section 210). FIG. 8 is a schematic of a catheter 100 having an asymmetrically-opening distal portion, according to aspects of the present invention. The figure shows that, in some examples described herein, the distal tip section 410 can have a substantially circular cross section with a center 432 radially offset from the longitudinal axis 111 of the elongate shaft 110, when in the expanded deployed configuration. This is shown in the examples in FIGS. 5 and 6 (i.e., distal tip section 410). The funnel profiles 434 in FIGS. 7 and 8, therefore, have different shapes in the expanded deployed configuration.

FIGS. 9A-9C are schematics illustrating a self-expanding catheter 100 used to capture a clot 40, according to aspects of the present invention. The examples shown in the schematics depict a self-expanding distal tip section 210. This description can apply to any distal tip section shape herein, wherein the distal tip section is heat set to have an expanded inner diameter 215 greater than the collapsed inner diameter 216. In FIG. 9A, the catheter 100 is deployed through a vessel 12 through a guide sheath 30 until the distal end 32 of the guide sheath 30 is proximate the clot 40. In FIG. 9B, the distal tip section 210 is then deployed from the guide sheath 30 and automatically expands into its expanded deployed configuration due to the heat setting into that configuration. In FIG. 9C, the distal tip section 210 is then advanced to the clot 40, suction is applied through the elongate shaft 110, and the clot can be retracted from the vessel 12.

FIG. 10 illustrates the distal portion of a clot retrieval catheter 100 with an expandable tip, according to aspects of the present invention. The example shown in FIG. 10 is substantially similar to the design shown in FIG. 3. However, in the design of FIG. 3, the distal tip section 310 can be considered a self-expanding design in that it is heat set into the expanded deployed configuration; the design in FIG. 10 instead includes a low-shear distal tip section 510 that expands upon contacting a clot 40. For example, a distal expansile section 530 of the distal tip section 510 can maintain a nominal tip inner diameter 516 until the distal end 514 of the distal tip section 510 contacts a clot 40. Upon contacting the clot 40, the distal tip section 510 can be advanced while suction is provided to surround the clot 40 for retrieval. Otherwise, the design shown in FIG. 10 can include a final crossover point 518, which is similar to final crossover point 318 in FIG. 3; can include two sets of opposing ribs 520 that flare outward upon contacting the clot 40, which are similar to the two sets of opposing ribs 320; can include a first rib 531, which is similar to first rib 330; can include a second rib 532, which is similar to second rib 332; a distally unconnected peak 528, which is similar to the distally unconnected peak 328; and the opposing ribs 520 can include the v-shaped pattern 529, which similar to the v-shaped pattern 329 in FIG. 3.

FIG. 11 illustrates the distal portion of a clot retrieval catheter 100 with an expandable tip, according to aspects of the present invention. The example shown in FIG. 11 is substantially similar to the design shown in FIG. 5. However, in the design of FIG. 5, the distal tip section 410 can be considered a self-expanding design in that it is heat set into the expanded deployed configuration; the design in FIG. 11 instead includes a low-shear distal tip section 610 that expands during clot 40 retrieval. For example, a distal expansile section 630 of the distal tip section 610 can maintain a nominal tip inner diameter 616 until the distal end 614 of the distal tip section 610 contacts a clot 40. Upon contacting the clot 40, the distal tip section 610 can be advanced while suction is provided to surround the clot 40 for retrieval. Otherwise, the design shown in FIG. 11 can include a bevel angle 617, which is similar to the acute angle 417 in FIG. 5; can include a final crossover point 618, which is similar to the final crossover point 418; can include a longitudinal array of offset hoops 620, which is similar to the longitudinal array of offset hoops 620; can include distally unconnected peaks 628, which are similar to the distally unconnected peaks 428. The longitudinal array of hoops 620 can define a mouth with a curvilinear profile 613. This curvilinear profile 613 can also be present for the opposing ribs 320 (see FIG. 3) and the array of offset hoops 420 (see FIG. 5).

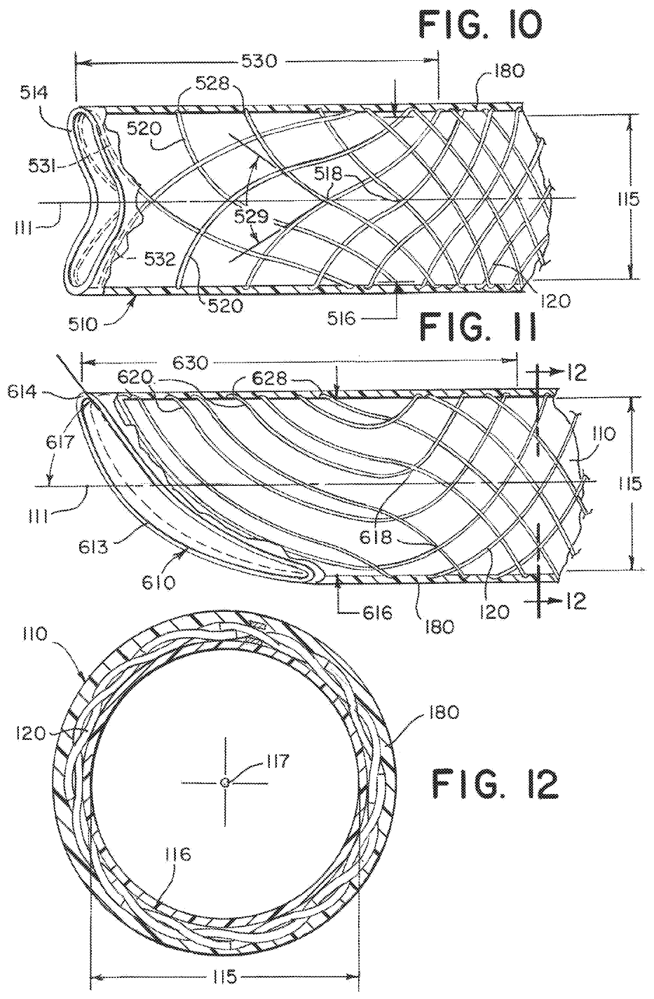

FIG. 12 is a cutaway view of the clot retrieval catheter 100 of FIG. 11, which shows the detail of an outer jacket 180, according to aspects of the present invention. Although the image is a cutaway of FIG. 11, it will be appreciated that the jacket 180 can be applied to any of the catheter 100 designs described herein. The jacket 180 can block proximal fluid from entering the expanded tip during aspiration and retrieval of the clot, allowing for more efficient direction of the aspiration force while preventing the distal migration of clot fragments or other debris during the procedure. In one example, the jacket 180 can be formed from a highly-elastic material such that the radial force exerted by expanding the expansile tip is sufficient to stretch the membrane to the funnel shape contours of the tip when in the expanded deployed configuration. One example can be using a ductile elastomer which has the advantages of being soft and flexible with resistance to tearing and perforation due to a high failure strain. Alternately, the jacket 180 can be loosely fitted to the catheter 100 and fold over the distal tip section edges so that the distal tip sections can move more freely when expanded and collapsed.

FIGS. 13A-13C are schematics illustrating a non-self-expanding catheter 100 used to capture a clot 40, according to aspects of the present invention. The examples shown in the schematics depict a non-self-expanding distal tip section 510. This description can apply to any distal tip section described herein, wherein the distal tip section is not heat set to have an expanded configuration but instead a distal expansile section (e.g., expansile section 430) expands to an expanded inner diameter 415 when contacting a clot 40. The distal tip section 410 can have a larger expanded inner diameter 415 when impinged radially by an ingested clot 40 in the expanded clot capture configuration and a smaller delivery inner diameter 115 in the delivery configuration. In FIG. 13A, the catheter is deployed through a vessel 12 through a guide sheath 30 until the distal end 32 of the guide sheath 30 is proximate the clot 40. At this point, the distal tip section 410 has a collapsed inner diameter 416. In FIG. 13B, the distal tip section 510 is then deployed from the guide sheath 30. In FIG. 13C, the distal tip section 510 is then advanced to the clot 40, wherein the distal tip section 510 expands as the distalmost tip 414 of the distal tip section 510 is advanced over the clot 40. Suction is applied through the elongate shaft 110, and the clot 40 can be retracted from the vessel 12.

The invention is not necessarily limited to the examples described, which can be varied in construction and detail. The terms “distal” and “proximal” are used throughout the preceding description and are meant to refer to a positions and directions relative to a treating physician. As such, “distal” or distally” refer to a position distant to or a direction away from the physician. Similarly, “proximal” or “proximally” refer to a position near or a direction towards the physician. Furthermore, the singular forms “a,” “an,” and “the” include plural referents unless the context clearly dictates otherwise.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

In describing example embodiments, terminology has been resorted to for the sake of clarity. As a result, not all possible combinations have been listed, and such variants are often apparent to those of skill in the art and are intended to be within the scope of the claims which follow. It is intended that each term contemplates its broadest meaning as understood by those skilled in the art and includes all technical equivalents that operate in a similar manner to accomplish a similar purpose without departing from the scope and spirit of the invention. It is also to be understood that the mention of one or more steps of a method does not preclude the presence of additional method steps or intervening method steps between those steps expressly identified. Similarly, some steps of a method can be performed in a different order than those described herein without departing from the scope of the disclosed technology.

What is claimed is:

1. A catheter comprising:
a proximal elongate shaft comprising a distal end, a longitudinal axis, and a shaft braid member comprising a plurality of braid wires; and
a distal tip section at the distal end of the elongate shaft, the distal tip section comprising a collapsed delivery configuration, an expanded deployed configuration, and a longitudinal array of hoops;
the plurality of braid wires of the proximal elongate shaft formed monolithically with the array of hoops of the distal tip section, and
each wire of the plurality of braid wires diverging from a final crossover point of the shaft braid member at the distal end of the proximal elongate shaft to form one of the hoops of the longitudinal array of hoops.

2. The catheter of claim 1, the distal tip section further comprising a collapsed inner diameter in the collapsed delivery configuration less than an expanded inner diameter in the expanded deployed configuration.

3. The catheter of claim 1, the wire of each hoop of the longitudinal array of hoops connected to the elongate shaft at the final crossover point by a pair of axially extending hoop runners.

4. The catheter of claim 3, each pair of axially extending hoop runners spaced evenly around the longitudinal axis.

5. The catheter of claim 3, each hoop of the longitudinal array of hoops diverging radially from a pair of hoop termini at the distal end of each pair of axially externing hoop runners to extend circumferentially around the tip section.

6. The catheter of claim 1, a first spacing between a pair of more proximal adjacent hoops of the longitudinal array of hoops being different than a second spacing between adjacent hoops in a more distal pair of adjacent hoops.

7. The catheter of claim 6, each hoop of the longitudinal array of hoops comprising a distally unconnected peak which moves distally when the distal tip section is folded to the collapsed delivery configuration.

8. The catheter of claim 1, each hoop of the longitudinal array of hoops defining a plane that is normal to the longitudinal axis of the elongate shaft.

9. A catheter comprising:
a proximal elongate shaft comprising a distal end, a longitudinal axis, and a shaft braid member comprising a plurality of braid wires; and
a distal tip section extending from the distal end of the elongate shaft, the distal tip section comprising two sets of opposing ribs;
a first rib from the two sets of opposing ribs formed from a first wire of the plurality of braid wires of the proximal elongate shaft and a second rib from the two sets of opposing ribs formed from a second wire of the plurality of braid wires of the proximal elongate shaft, the first rib being spaced approximately 180 degrees about the longitudinal axis from the second rib.

10. The catheter of claim 9, the distal tip section further comprising a delivery configuration and a clot capture configuration;
the distal tip section having a smaller delivery inner diameter in the delivery configuration and a larger expanded inner diameter when impinged radially by an ingested clot in the clot capture configuration.

11. The catheter of claim 9, the distal tip section further comprising a collapsed delivery configuration having a collapsed inner diameter and an expanded deployed configuration heat set to have an expanded inner diameter greater than the collapsed inner diameter.

12. The catheter of claim 9, each rib of the two sets of opposing ribs comprising a distally unconnected peak.

13. The catheter of claim 9, the two sets of opposing ribs formed monolithically with the plurality of braid wires of the shaft braid member.

14. The catheter of claim 9, each wire of the plurality of braid wires diverging radially from a final crossover point to form a v-shaped pattern.

15. A catheter comprising:
a proximal elongate shaft comprising a distal end, a longitudinal axis, and a shaft braid member comprising a plurality of braid wires;
a distal tip section at the distal end of the elongate shaft, the distal tip section comprising a longitudinal array of offset hoops defining a beveled plane; and
a distal outer jacket surrounding the longitudinal array of offset hoops;
the plurality of braid wires of the proximal elongate shaft formed monolithically with the array of offset hoops, each wire of the plurality of braid wires diverging from a final crossover point to extend circumferentially around the tip section.
the beveled plane crossing the longitudinal axis at an acute angle.

16. The catheter of claim 15, the distal tip section further comprising a collapsed delivery configuration having a collapsed inner diameter and an expanded deployed configuration having an expanded inner diameter heat set to be greater than the collapsed inner diameter.

17. The catheter of claim 15, the distal tip section further comprising a larger expanded inner diameter when impinged radially by an ingested clot in an expanded clot capture configuration and a smaller delivery inner diameter in a delivery configuration.

18. The catheter of claim 17, wherein in the expanded clot capture configuration, the distal tip section further comprises a substantially circular cross section with a center radially offset from the longitudinal axis of the elongate shaft.

19. The catheter of claim 15, the array of offset hoops comprising distally unconnected peaks which move distally when the distal tip section is folded to a collapsed delivery configuration.

20. The catheter of claim 15, the array of offset hoops following a curvilinear profile circumferentially around the tip section.
